(19) United States
(12) Plant Patent Application Publication
(10) Pub. No.: US 2002/0010955 P1
(43) Pub. Date: Jan. 24, 2002

(54) Chrysanthemum named 'Sunny Elite Reagan'

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/902,749

(22) Filed: Jul. 12, 2001

(30) Foreign Application Priority Data
May 3, 1999 (NL) CHR 3121
Jul. 28, 2000 (EP) 2000/1173
Mar. 17, 2000 (PL) O 800

(51) Int. Cl. A01H 5/00

(52) U.S. Cl. PLT/295

(57) ABSTRACT

A Chrysanthemum plant named ‘Sunny Elite Reagan’ characterized by its medium sized blooms with yellow ray-florets and yellow-green disc florets.

### RELATED CULTIVARS

‘Sunny Elite Reagan’ is related to ‘White Elite Reagan’ (#______) and the co-pending application of ‘Cream Elite Reagan’ (#______). ‘Sunny Elite Reagan’ and ‘Cream Elite Reagan’ are both natural mutants of ‘White Elite reagan’

### BACKGROUND OF THE INVENTION

‘Sunny Elite Reagan’ is a product of a breeding and selection program which had the objective of fmding color mutants of ‘White Elite Reagan’. The new plant of the present invention comprises a new and distinct cultivar of chrysanthemum plant that is a natural occuring sport of a parent chrysanthemum named ‘White Elite Reagan’ ( #______). A comparison with Parent chrysanthemum ‘White Elite Reagan’ is also made in this application. The new cultivar was discovered as a whole plant mutation in April 1999 by Rob Noodelijk in a controlled environment (greenhouse) in Rijsenhout Holland. The first act of asexual reproduction of ‘Sunny Elite Reagan’ was accomplished when vegetative cuttings were taken from the initial selection in June 1999 in Rijsenhout Holland.

### SUMMARY OF THE INVENTION

The present invention is a new and distinct variety of chrysanthemum bearing medium sized blooms with yellow ray-florets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

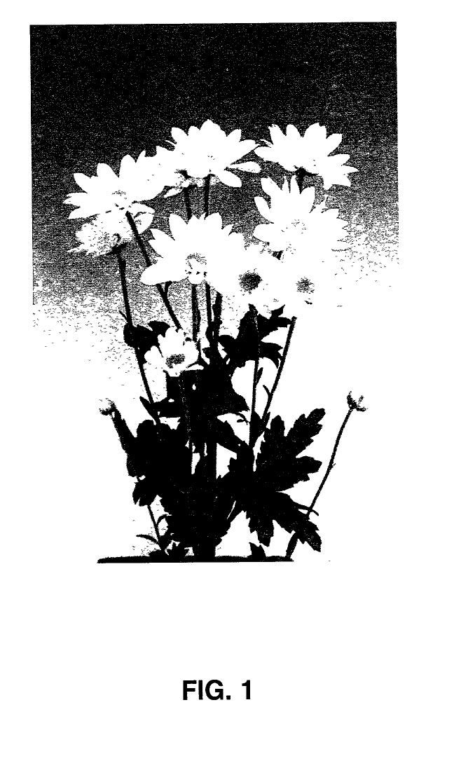

FIG. 2 shows the various stages of bloom of the new cultivar.

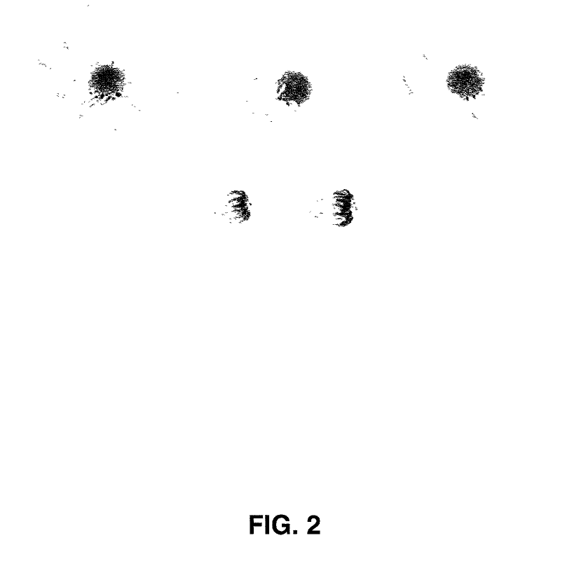

FIG. 3 shows the foliage of the new cultivar.

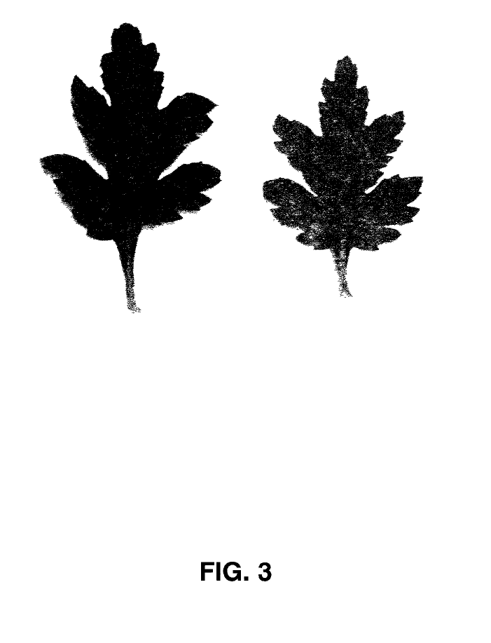

### DESCRIPTION OF THE INVENTION

This new variety of chrysanthemum is of the botanical classification dendranthema grandiflora. The observations and measurements were gathered from plants grown in April/May in a greenhouse in Rijsenhout Holland in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 50 days. After the long day period to flowering growth retardants were applied 2 to 3 times in an average dose of 2.5 gram/liter water. No tests were done on disease or insect resistance or susceptibility. This new variety produces medium sized blooms with yellow ray-florets and yellow-green disc-florets blooming on the plant for 5 weeks. This new variety of chrysanthemum has been found to retain its distinctive characteristics throughout successive propagations however the phenotype may vary significantly with variations in environment such as light intensity and temperature. To show the phenotype as described ‘Sunny Elite Reagan’ can be planted without assimilation lightning (high pressure sodium lamps) between week 50 and week 40 of the next year under greenhouse conditions in Holland. With assimilation light (minimum level 2500 lux) it can be planted year round under greenhouse conditions in Holland.

From the cultivars known to inventor the most similar existing cultivar in comparison to ‘Sunny Elite Reagan’ is ‘White Elite Reagan’ and ‘Sunny Reagan (U.S. Plant Pat. No. 9928). When ‘White Elite Reagan’ and ‘Sunny Elite Reagan’ are being compared the following differences and similarities are noticed: The difference of ‘White Elite Reagan’ and ‘Sunny Elite Reagan’ is the flower color for which characteristics ‘Sunny Elite Reagan’ has been selected out of ‘White Elite Reagan’. All other characteristics of ‘White Elite Reagan’ and ‘Sunny Elite Reagan’ are similar.

The differences of ‘Sunny Elite Reagan’ and ‘Sunny Reagan’ are: (1) Disc flower color and size. The flower disc of ‘Sunny Elite Reagan’ is more green and smaller. (2) Growth rate. The growth of ‘Sunny Elite Reagan’ is more vigorous. (3) Response time. The response time of ‘Sunny Elite Reagan’ is 3 till 4 days shorter.

The following is a description of the plant and characteristics that distinguish ‘Sunny Elite Reagan’ as a new and distinct variety.

The color designations are taken from the plant itself. Accordingly, any discrepancies between the color designations and the colors depicted in the photographs are due to photographic tolerances. The color chart used in this description is: The Royal Horticultural Society color chart, edition 1995.

TABLE 1
Botanical Description of
CULTIVAR ‘Sunny Elite Reagan’

| | |
|---|---|
| Bud | |
| Size | Medium, cross-section +/− 1.0 cm |
| | height +/− 0.8 cm |
| Outside Color | Yellow 9 A |
| Involucral bracts | 2 rows, length 7 mm, width 3 mm |
| Involucral bracts among | Not present |
| disc-florets | |
| Involucral bracts color | Green 138 B |
| Bloom | |
| Type | Single |
| Height | Flat |
| Size | Medium |
| Fully Expanded | 6.0-7.0 cm |
| Number of blooms per | Average of 11-12 |
| single stem | |
| Seeds | Not produced |
| Performance (blooming period) | 26-28 days |
| Fragrance | Typical chrysanthemum |
| Color | |
| Center of the flower | Immature yellow-green 144 B, mature |
| (disc-florets) | yellow-green 144 B to yellow 3 A |
| | along the border |
| Color of upper surface of | Yellow 5 A |
| the majority of the ray-florets | |
| Color of the lower surface of | Yellow 5 C |
| the majority of the ray-florets | |
| Tonality from Distance | A spray mum with yellow flowers and a |
| | yellow-green disc |
| Discoloration to color | Yellow 4 A |
| Ray florets | |
| Texture | Upper and under side smooth |
| Number | 20-22 |
| Cross-section | Flat, sometimes convex |
| Longitudinal axis of majority | Straight, sometimes reflexing |
| Length of corolla tube | Very short to short |
| Ray-floret length | 3.5 cm |
| Ray-floret width | 1.2 cm |
| Ratio length/width | Low to medium |
| Shape of tip | Pointed |
| Disc florets | |
| Disc diameter | Medium to small, 1.0-1.2 cm |
| Distribution of disc florets | Numerous, clearly visible at all |
| | stages of flowering |
| Type | Tubular |
| Color | Yellow-green 144 A |
| Receptacle shape | Conical raised |
| Reproductive Organs | |
| Stamen (present in disc | Yellow-green, thin 3 mm in length |
| florets only) | |
| Stamen color | Yellow-green 150 B |
| Pollen | Appears at a very late stage of |
| | flowering, |
| Pollen color | Close to yellow 12 A |
| Styles (present in both | Green, thin |
| ray and disc florets) | |
| Style color | Green 143 B |
| Style Length | 4 mm |
| Stigmas | Yellow-green |
| Stigma Width | 1 mm |
| Ovaries | Enclosed in calyx |
| Plant | |
| Form | A spray mum meant for erect culture |
| Growth habit | Upright |
| Growth rate | Vigorous |
| Height | 105-130 cm |
| internodes | 3-3.5 cm |
| Spray formation | Corymbiform (funnel-shaped with a |
| | little round to flat umbel on top) |
| Peduncle length | Near the top 14 cm, near the middle |
| | 21 cm, near the bottom 26 cm |
| Peduncle color | Green 138 B |
| Peduncle, attachment | Good, not brittle |
| Peduncle, angle with stem | Small, 30° |
| Stem Color | Green 137 A |
| Stem Strength | Medium |
| Stem Brittleness | Absent |
| Stem Anthocyanin Coloration | Absent |
| Flowering Response | 50 days |
| (photo-periodic controlled | |
| crop, not natural season) | |
| Foliage | |
| Color | Upper side Under side |
| Size | Medium; length +/− 12 cm, |
| | width +/8 cm |
| Quantity (number per | 22-28 |
| single stem) | |
| Shape | Ovate |
| Texture upper side | Scabrous |
| Texture under side | Slightly pubescent |
| Venation arrangement | Pinnate |
| Shape of the margin | Lobed |
| Shape of Base of Sinus Between | Round |
| Lateral Lobes | |
| Margin of Sinus Between | Diverging |
| Lateral | |
| Lobes | |
| Shape of Base | Truncate, sometimes asymmetrical |
| Apex | Mucronate |

### TABLE 2

### Differences with the Comparison Varieties

TABLE 2
Differences with the comparison Varieties

| | ‘Sunny | ‘White | ‘Sunny |
|---|---|---|---|
| | Elite Reagan’ | Elite Reagan’ | Reagan’ |
| Color of the ray-florets | Yellow 5 A | White 155 D | Yellow 5 A |
| Flower disc size | 1.0-1.2 cm | 1.0-1.2 cm | 1.5 cm |
| Flower disc color | Yellow-green | Yellow-green | Yellow-green |
| immature | 144 A | 144 A | 151 C |
| Growth rate | vigorous | vigorous | Strong |
| Peduncle, angle with | 30° | 30° | 30-35° |
| stem | | | |
| Response time in days | 50 | 49 | 53-54 |

I claim:

1. A new and distinct variety of chrysanthemum plant as described and illustrated.
